(19) United States
(12) Patent Application Publication
(10) Pub. No.: US 2026/0355080 A1
(43) Pub. Date: Nov. 12, 2026

(54) COSMETIC COMPOSITION FOR PREVENTING OR AMELIORATING SKIN DISEASES, COMPRISING MORUS ALBA-DERIVED COMPOUND AS ACTIVE INGREDIENT

(71) Applicant: DONGGUK UNIVERSITY INDUSTRY-ACADEMIC COOPERATION FOUNDATION, Seoul (KR)

(72) Inventors: Ai-Young LEE, Seoul (KR); Young-Won CHIN, Seoul (KR); Ha Jung KIM, Goyang-si (KR); Nan-Hyung KIM, Goyang-si (KR)

(73) Assignee: DONGGUK UNIVERSITY INDUSTRY-ACADEMIC COOPERATION FOUNDATION, Seoul (KR)

(21) Appl. No.: 19/108,079

(22) PCT Filed: Aug. 31, 2023
(86) PCT No.: PCT/KR2023/012954

§ 371 Date: Feb. 28, 2025

(30) Foreign Application Priority Data
Aug. 31, 2022 (KR) 10-2022-0109615
Aug. 28, 2023 (KR) 10-2023-0112873

(51) Int. Cl. A61K 8/37; A61K 8/34; A61Q 19/02; A61Q 19/08

(52) U.S. Cl. CPC A61K 8/37; A61K 8/347; A61Q 19/02; A61Q 19/08; A61K 2800/92

(57) ABSTRACT

The present invention relates to a cosmetic composition for preventing or ameliorating skin diseases, comprising a Morus alba-derived compound as an active ingredient, and a specific compound derived from Morus alba fractions has been confirmed to exhibit anti-aging and whitening effects at the same time, and thus, can be useful as compositions for preventing, treating, or ameliorating skin diseases.

### TECHNICAL FIELD

The present disclosure relates to a cosmetic composition for preventing or ameliorating a skin disease, including a Morus alba-derived compound as an active ingredient.

### BACKGROUND ART

In recent years, aesthetic factors have been recognized as an important social competitiveness due to the improvement of living standards, the increase in income levels, and the increased social interest in beauty, and the desire to maintain a more youthful and beautiful appearance has also become stronger, such that the demand and interest in cosmetic products continue to grow. In the case of functional cosmetic products, multifunctional cosmetic products that have various skin ameliorating effects such as anti-aging, UV protection, wrinkle amelioration, and whitening are widely used, with a high demand particularly for whitening cosmetics in the Asian market.

Aging is a phenomenon that the body functions of living organisms deteriorate over time. Cellular senescence is manifested by the loss of ability of cells to divide. In general, cellular senescence includes senescence mechanisms dependent on a retinoblastoma (Rb) protein, senescence caused by the loss of telomeres that play a role in protecting chromosomes, aging due to antioxidant stress, and aging induced by DNA damage. Thereamong, skin aging is classified into intrinsic aging (internal aging) and extrinsic aging (external aging). Intrinsic aging, a phenomenon that occurs naturally over time due to the influence of genetic factors regardless of the external environment, is difficult to artificially control, and functional changes appear rather than visual and morphological changes. On the other hand, extrinsic aging is a phenomenon caused by external environmental factors such as chronic exposure to sunlight, and the artificial control thereof is relatively easy with distinct structural and physiological changes. Since it is mainly caused by exposure to the sunlight (ultraviolet rays), it is also called photoaging. In aged skin, increased drug permeability, increased susceptibility to irritant contact dermatitis, worsening of dry skin, itching, wrinkles, and pigmentation abnormalities (age spots, melasma, etc.) occur.

Human's skin color largely depends on the amount of melanin, hemoglobin, and carotene, and thereamong, the quinone complex called melanin, which is synthesized by melanoblasts that are present in the basal cell layer of the epidermis, plays the most important role. Melanin not only determines the human's skin color but also plays a role in protecting the skin, but when overproduced in the skin, pigmentation occurs in the skin, resulting in darkening of skin, melasma, and freckles. The cause of melanin production is a combination of external environmental changes such as exposure to ultraviolet rays, air pollutants, medications, topical irritating skin agents, and stress, as well as internal factors such as hormonal imbalances, inflammatory response factors, and cytokines, but the most important of these is exposure to ultraviolet rays.

As it has been revealed that tyrosinase plays a decisive role in the synthesis of melanin, generally used are methods to reduce the melanin production by blending substances with an inhibitory effect on tyrosinase activity in cosmetics or by inhibiting some reactions during the melanin production process. Typical materials that inhibit the tyrosinase activity include hydroquinone, azelaic acid, and kojic acid, and are also used in the form of external applications in the combination of hydroquinone+tretinoin+steroids, but their effects are not yet satisfactory.

Therefore, research is actively underway to develop materials that exhibit various skin ameliorating effects such as anti-aging, UV protection, wrinkle amelioration, and whitening.

### DISCLOSURE OF THE INVENTION

### Technical Goals

An object of the present disclosure is to provide a cosmetic composition for preventing or ameliorating a skin disease, including a Morus alba-derived compound as an active ingredient.

Another object of the present disclosure is to provide a health functional food composition for preventing or ameliorating a skin disease, including the compound as an active ingredient.

Still another object of the present disclosure is to provide a pharmaceutical composition for preventing or treating a skin disease, including the compound as an active ingredient.

### Technical Solutions

To achieve the above objects, the present disclosure provides a cosmetic composition for preventing or ameliorating a skin disease, including a compound represented by the following Chemical Formula 1 or a pharmaceutically acceptable salt thereof as an active ingredient.

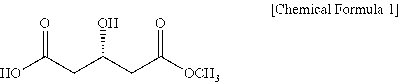 [SMILES: COC(=O)C[C@@H](O)CC(=O)O]

In addition, the present disclosure provides a health functional food composition for preventing or ameliorating a skin disease, including the compound or a foodologically acceptable salt thereof as an active ingredient.

In addition, the present disclosure provides a pharmaceutical composition for preventing or treating a skin disease, including the compound or a pharmaceutically acceptable salt thereof as an active ingredient.

### Advantageous Effects

According to the present disclosure, it was found that a specific compound derived from a Morus alba fraction exhibits both anti-aging and whitening effects, such that it may be useful as a composition for preventing, treating, or ameliorating a skin disease.

### BRIEF DESCRIPTION OF DRAWINGS

FIG. 1 shows a result of analyzing cytotoxicity of a M55 compound, which is a compound derived from a Morus alba-derived fraction, in melanocytes.

FIG. 2 shows a result of analyzing an effect of the M55 compound on tyrosinase expression in melanocytes.

FIG. 3 shows results of analyzing an effect of a M55 compound on tyrosinase expression in three types of cell models constructed in Example 2-1.

FIG. 4 shows a result of analyzing an effect of a M55 compound on p16 (Ink4a) expression in keratinocytes.

### BEST MODE FOR CARRYING OUT THE INVENTION

Hereinafter, the present disclosure will be described in more detail.

The present disclosure provides a cosmetic composition for preventing or ameliorating a skin disease, including a compound represented by the following Chemical Formula 1 or a pharmaceutically acceptable salt thereof as an active ingredient.

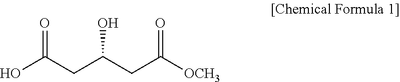 [SMILES: COC(=O)C[C@@H](O)CC(=O)O]

The compound or a pharmaceutically acceptable salt thereof may be derived from Morus alba.

In addition, the compound or a pharmaceutically acceptable salt thereof may exhibit anti-aging or whitening effects and suppress expression of p16 (Ink4a) or tyrosinase.

The skin disease may be a skin aging disease; or a skin disease caused by overproduction of melanin.

The skin aging disease may be one or more selected from the group consisting of skin wrinkles, photosenescence, melanosis, skin pigmentation, skin burns, skin inflammation, and skin cancer, and the skin disease caused by overproduction of melanin may be one or more selected from the group consisting of melasma, freckles, blemishes, age spots, and Ota nevus, but are not limited thereto.

The term “whitening” as used herein refers to an action of suppressing, inhibiting, or alleviating the hyperpigmentation of melanin, and preventing the darkening of skin caused by pigmentation so as to improve skin problems.

The term “cosmetic” as used herein may include functional cosmetics that have effects such as amelioration of skin diseases, and the functional cosmetics have professional functionality with bioactive efficacy and effects emphasized unlike general cosmetics and refer to cosmetics with specific efficacy and effects emphasized such as amelioration of a skin disease as defined in the Cosmetics Act.

The term “including as an active ingredient” may refer to inclusion in an effective amount that may derive an effect of ameliorating skin diseases.

Specifically, the cosmetic composition may be prepared in a general emulsifying formulation and a solubilized formulation. For example, it may be formulated into lotions such as softening lotions or nourishing lotions; emulsions such as facial lotions and body lotions; creams such as nourishing creams, moisturizing creams, and eye creams; essence; cosmetic ointments; spray; gel; pack; sunscreen; makeup base; foundations in a liquid type, solid type, or spray type; powder; makeup removers such as cleansing creams, cleansing lotions, and cleansing oils; or cleansers such as cleansing foam, soaps, and body washes, but is not limited thereto. In addition, the external skin agent may be formulated as an ointment, patch, gel, cream, or nebulizer, but is not limited thereto.

For each formulation, in addition to the essential ingredients, the cosmetic composition may be appropriately blended with other ingredients, within a range that it does not interfere with the purpose according to the present disclosure depending on the type of formulation or the purpose of use.

The cosmetic composition may include a conventionally acceptable carrier, for example, it may be appropriately blended with oil, water, surfactants, moisturizers, low-grade alcohol, thickeners, chelating agents, colors, preservatives, and fragrances, but is not limited thereto.

The acceptable carrier may vary depending on the formulation. For example, animal oils, vegetable oils, waxes, paraffins, starches, tracanths, cellulose derivatives, polyethylene glycol, silicones, bentonite, silica, talc, zinc oxide, or extracts thereof may be used as carrier components when formulated into ointments, pastes, creams, or gels.

When the cosmetic composition is formulated as the powder or spray, lactose, talc, silica, aluminum hydroxide, calcium silicate, polyamide powder, or an extract thereof may be used as the carrier component, and in the case of the spray, a booster such as chlorofluorohydrocarbon, propane, butane, or dimethyl ether may be additionally included.

When the cosmetic composition is formulated as the solution or emulsion, solvents, solubilizers, or emulsifiers may be used as the carrier component, for example water, ethanol, isopropanol, ethyl carbonate, ethyl acetate, benzyl benzoate, propylene glycol, and 1,3-butyl glycol oil may be used, in particular, cottonseed oil, peanut oil, corn germ oil, olive oil, castor oil, and sesame seed oil, glycerol aliphatic esters, polyethylene glycol, or fatty acid esters of sorbitan may be used.

When the cosmetic composition is formulated as the suspension, water, liquid diluents such as ethanol or propylene glycol, ethoxylated isostearyl alcohol, suspension agents such as polyoxyethylene sorbitol ester and polyoxyethylene sorbitan ester, microcrystalline cellulose, aluminum metahydroxide, bentonite, agar, or tracanth may be used as the carrier component.

When the cosmetic composition is formulated into soap, alkali metal salts of fatty acids, fatty acid hemiester salts, fatty acid protein hydrolyzate, isethionate, lanolin derivatives, aliphatic alcohol, vegetable oil, glycerol, and sugar may be used as the carrier components.

The cosmetic composition may additionally contain supplements that are commonly used in the field of cosmetology or dermatology, such as fatty substances, organic solvents, solubilizers, thickeners, gelling agents, emollients, antioxidants, suspensions, stabilizers, foaming agents, aromatic agent, surfactants, water, ionic or non-ionic emulsifiers, fillers, metal ion sequestering agent, chelating agents, preservatives, blockers, humectants, essential oils, dyes, pigments, hydrophilic or lipophilic activators, and any other ingredients commonly used in cosmetics, that are conventionally used in the art depending on the quality or function of final products.

However, the supplement and a mixing ratio thereof may be appropriately selected so as not to affect the desirable properties of the cosmetic composition according to the present disclosure.

In addition, the present disclosure provides a health functional food composition for preventing or ameliorating a skin disease, including a compound represented by the following Chemical Formula 1 or a foodologically acceptable salt thereof as an active ingredient.

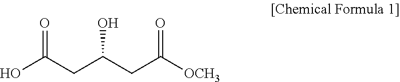 [SMILES: COC(=O)C[C@@H](O)CC(=O)O]

The present disclosure may be used generally as a commonly used food product.

The food composition of the present disclosure may be used as a health functional food. The term “health functional food” as used herein refers to food manufactured and processed with raw materials or ingredients having useful functionality for the human body in accordance with the Health Functional Food Act, and the term “functionality” as used herein refers to the intake to derive effectiveness in health care such as physiological actions or regulation of nutrients for the structure and function of the human body.

The health functional food composition may include conventional food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills. For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action of suppressing or delaying skin diseases by administration of the composition according to the present disclosure.

The term “treatment” as used herein refers to any action that ameliorates or favorably changes the symptoms of skin diseases by administration of the composition according to the present disclosure.

The term “amelioration” as used herein refers to any action that makes a bad state of skin diseases better by administration of the composition according to the present disclosure.

In addition, the present disclosure provides a pharmaceutical composition for preventing or treating a skin disease, including a compound represented by the following Chemical Formula 1 or a pharmaceutically acceptable salt thereof as an active ingredient.

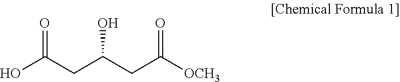 [SMILES: COC(=O)C[C@@H](O)CC(=O)O]

The pharmaceutical composition of the present disclosure may be prepared in the form of a unit dose by formulation using a pharmaceutically acceptable carrier or prepared by encapsulating into a multi-capacity container, in accordance with a method that may be easily carried out by a person skilled in the art to which the disclosure pertains.

The pharmaceutically acceptable carriers are commonly used in preparation, including, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further include lubricants, humectants, sweeteners, flavoring agents, emulsifiers, suspensions, and preservatives, in addition to the above ingredients.

In the present disclosure, the content of the additives included in the pharmaceutical composition is not particularly limited and may be appropriately adjusted within a content range used in the conventional preparation.

The pharmaceutical composition may be formulated in the form of one or more external skin agents selected from the group consisting of, but are not limited to, injectable formulations such as aqueous solutions, suspensions, and emulsions, pills, capsules, granules, tablets, creams, gels, patches, sprays, ointments, plasters, lotions, liniments, pastas, and cataplasmas.

The pharmaceutical composition of the present disclosure may include a pharmaceutically acceptable carrier and diluent that are additionally present for formulation. The pharmaceutically acceptable carriers and diluents include, but are not limited to, excipients such as starch, sugars, and mannitol, fillers and extenders such as calcium phosphate, cellulose derivatives such as carboxymethylcellulose and hydroxypropylcellulose, binders such as gelatin, alginate, and polyvinyl pyrrolidone, lubricants such as talc, calcium stearate, hydrogenated castor oil, and polyethylene glycol, disintegrating agents such as povidone and crospovidone, and surfactants such as polysorbate, cetyl alcohol, and glycerol. The pharmaceutically acceptable carriers and diluents may be biologically and physiologically friendly to a subject. Examples of diluents may include, but are not limited to, brine, water-soluble buffers, solvents, and/or dispersion media.

The pharmaceutical composition of the present disclosure may be administered orally or parenterally (e.g., applied intravenously, subcutaneously, intraperitoneally, or topically) depending on the desired method. When administered orally, it may be formulated as tablets, troches, lozenges, water-soluble suspensions, oil-based suspensions, powder preparation, granules, emulsions, hard capsules, soft capsules, syrups, or elixirs. When administered parenterally, it may be formulated as injection solutions, suppositories, powders for respiratory inhalation, aerosols for sprays, ointments, powders for application, oils, or creams.

The dosage of the pharmaceutical composition of the present disclosure may vary depending on the patient's condition, weight, age, sex, health status, dietary constitution specificity, nature of the preparation, severity of disease, administration time for composition, method of administration, duration or interval of administration, excretion rate, and drug form, and may be appropriately selected by those skilled in the art. For example, it may range from about 0.1 to 10,000 mg/kg, but is not limited thereby, and it may be administered once or several times a day.

The pharmaceutical composition may be administered orally or parenterally (e.g., applied intravenously, subcutaneously, intraperitoneally, or topically) depending on the desired method. The pharmaceutically effective amount and effective dosage of the pharmaceutical composition of the present disclosure may vary by the preparation method of the pharmaceutical composition, type of administration, administration time, and administration route, and a person skilled in the art may easily determine and prescribe the effective dosage for the desired treatment. The administration of the pharmaceutical composition of the present disclosure may be conducted once a day or in several divided doses.

### MODES FOR CARRYING OUT THE INVENTION

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### [Experimental Example 1] Cell Culture

### 1-1. Keratinocytes

Keratinocytes used were isolated from normal adult skin tissues obtained through repeated cesarean section and circumcision. The cells were dispensed into EpiLife Medium, and bovine pituitary extract (BPE), bovine insulin (BI), hydrocortisone, human epidermal growth factor (EGF), and bovine transferrin (BT) were added and cultured. The cells that had undergone the 3^{rd}~5^{th} passage culture were used in the experiment. The pituitary extract, bovine insulin, hydrocortisone, human epidermal growth factor, and bovine transferrin were used by purchasing from Invitrogen.

### 1-2. Melanocytes

Melanocytes used were isolated from normal adult skin tissues obtained through repeated cesarean section and circumcision. Dispase 2.4U (#295 825; Roche, Mannheim, Germany) was treated for 1 hour to separate the epidermis, and 0.05% trypsin was treated for 10 minutes to isolate melanocytes. Afterwards, the cells were suspended in Medium 254, and the bovine pituitary extract (BPE), fetal bovine serum (FBS), hydrocortisone, basic fibroblast growth factor (bFGF), bovine transferrin (BT), heparin, and phorbol 12-myristate13-acetate (PMA, Invitrogen) were added and cultured. The cells that had undergone the 3^{rd}~5^{th} passage culture were used in the experiment.

### [Example 1] Screening of Substances Having Skin Whitening Effects

In order to identify substances that exhibit skin whitening effects, the following 136 candidate materials were prepared. The following candidate materials used were distributed by Professor Young Won Jin from Natural Products Research Institute in Seoul National University.
1) 35 compounds (T1~T35) derived from a Tribulus terrestris fraction
2) 63 compounds (M1~M63) derived from a Morus alba fraction
3) Other 38 compounds (CZ1~CZ38)

### 1-1. Cytotoxicity Assay

In order to identify cytotoxicity of the 136 candidate materials, MTT assays were performed. Melanocytes were treated with the candidate materials by concentration (10, 50, and 100 μM) for 48 hours, and the cell viability was measured to determine the concentration range at which no cytotoxicity of each candidate material is observed.

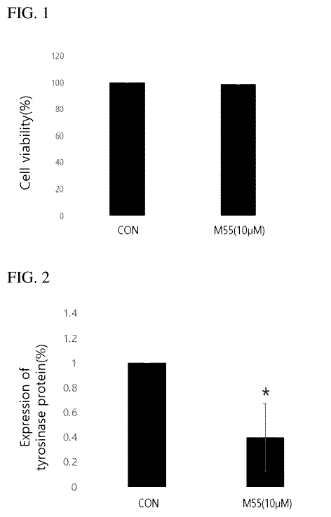

As a result, the concentration at which no cytotoxicity was observed for each candidate material was determined, and as shown in FIG. 1, the M55 compound, one of the fractions extracted from Morus alba, did not show cytotoxicity up to the treatment concentration of 10 μM.

### 1-2. Analysis on the Skin Whitening Effect

In order to identify the skin whitening effect of the 136 candidate materials, Western blot was performed. After treating melanocytes with the candidate material at a concentration at which no cytotoxicity was shown in the melanocytes identified in Example 1-2 for 48 hours, intracellular tyrosinase expression was measured. The experiment was repeated 4 times or more.

As a result, tyrosinase expression was significantly reduced in the group treated with 16 candidate materials, and as shown in FIG. 2, thereamong, tyrosinase expression was significantly reduced in the group treated with the M55 compound (10 μM) compared to the control group (CON; no candidate material treated group).

### [Example 2] Analysis on Substances that Show Skin Whitening and Anti-Aging Effects

### 2-1. Construction of Cell Models

In order to identify the substance that exhibits both skin whitening and anti-aging effects among the 16 candidate materials in the Example 1-2, as shown below, 3 types of cell models were first constructed.
1) Cell models by co-culture of ARG2 overexpressing keratinocytes and normal melanocytes
2) Cell models by co-culture of CDH11 overexpressing keratinocytes and melanocytes
3) Cell models by co-culture of GDA overexpressing keratinocytes and melanocytes

Keratinocytes were seeded in a culture container at 2×10⁵/ml, after 24 hours, pCMV plasmids containing one of three genes [arginase 2 (ARG2, NCBI Gene ID: 384), cadherin 11 (CDH11, NCBI Gene: 1009), and guanine deaminase (GDA, NCBI Gene: 9615)] were transfected into cells using Lipofectamine 2000 to induce overexpression, and after 4 hours of culture with Dulbecco's Modified Essential Medium (DMEM) culture medium without supplements, 0.5×10⁵/ml melanocytes were mixed with the keratinocyte culture medium containing supplements and co-cultured in the same culture container. After 24 hours, the keratinocyte culture medium without supplements was replaced, and after 5 hours, the normal melanocytes were treated with the 16 candidate materials for 48 hours. The cell model, by co-culture of keratinocytes overexpressing each of the three control genes (ARG2, CDH11, and GDA) and normal melanocytes, was set as the control group, and the keratinocytes overexpressing the control gene for each of the three genes were prepared by transfecting keratinocytes with the pCMV plasmid containing an empty vector without insertion of the three genes.

### 2-2. Analysis of Skin Whitening and Anti-Aging Effects

In order to identify the skin whitening and anti-aging effects of 16 candidate materials in Example 1-2, for the skin whitening effect, each of the three types of cell models constructed in the Example 2-1 was treated with the 16 candidate materials at concentrations at which no cytotoxicity is shown in the melanocytes identified in Example 1-1 for 48 hours, and then Western blot was performed to measure the expression of tyrosinase. The experiment was repeated 4 times, and P<0.05 was determined to be significant. In addition, for analysis on the anti-aging effect, the keratinocytes were treated with the above 16 candidate materials for 48 hours each, and then Western blot was performed to measure the expression of p16 (Ink4a). The experiment was repeated 4 times, and P<0.05 was determined to be significant.

As a result, out of the 16 candidate materials, skin whitening and anti-aging effects were shown together in the group treated with the 5 candidate materials, and it was determined that M55 compound was also included in the 5 candidate materials.

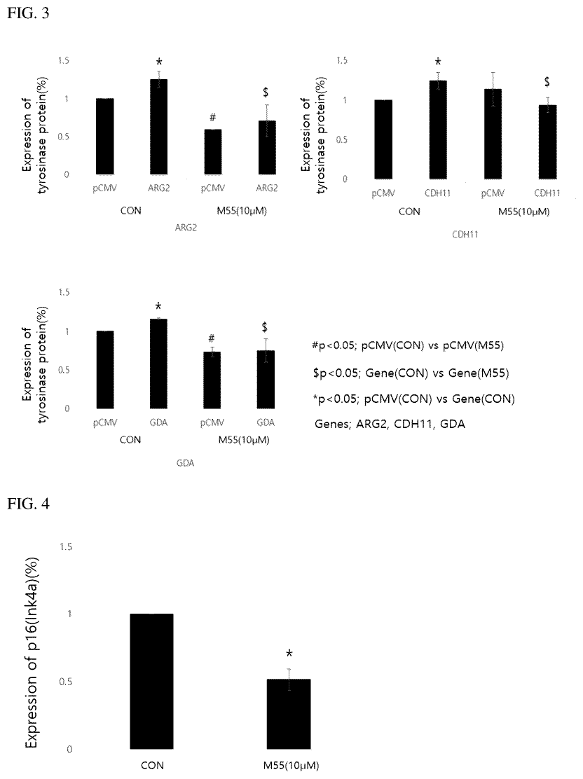

Specifically, for the skin whitening effect, as shown in FIG. 3, in the case of the control group (CON; no material treated group; the group treated solely with solvents that dissolve the material), tyrosinase expression was significantly increased in the ARG2 overexpressing co-cultured cell model (ARG2) compared to the control gene overexpressing co-cultured cell model (pCMV). The M55 treated group (M55, 10 μM) showed a significant decrease in tyrosinase expression compared to the control group.

In addition, as shown in FIG. 3, in the control group (CON; no material treated group; the group solely treated with the solvents that dissolve materials), tyrosinase expression was significantly increased in the CDH11 overexpressing co-cultured cell model (CDH11) compared to the control gene overexpressing co-cultured cell model (pCMV). In the case of the control gene overexpressing co-cultured cell model, the expression was increased in the M55 treated group (M55, 10 μM) compared to the control group, while, in the case of the CDH11 overexpressing co-cultured cell model, the tyrosinase expression was reduced in the M55 treated group compared to the control group.

In addition, as shown in FIG. 3, in the control group (CON; no material treated group; the group solely treated with the solvents that dissolve materials), tyrosinase expression was significantly increased in the GDA overexpressing co-cultured cell model (GDA) compared to the control gene overexpressing co-cultured cell model (pCMV). The M55 treated group (M55, 10 μM) showed a significant decrease in the tyrosinase expression compared to the control group.

In addition, in the case of anti-aging effects, as shown in FIG. 4, p16 expression was significantly reduced in the M55 treated group (M55, 10 μM) compared to the control group (CON; no candidate material treated group).

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the appended claims and their equivalents.

1-9. (canceled)

10. A method for preventing or ameliorating a skin disease, comprising administering cosmetically effective amount of a cosmetic composition comprising a compound represented by the following Chemical Formula 1 or a pharmaceutically acceptable salt thereof as an active ingredient to a subject in need of preventing or ameliorating the skin disease:

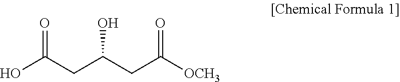 [SMILES: COC(=O)C[C@@H](O)CC(=O)O]

11. The method of claim 10, wherein the compound or the pharmaceutically acceptable salt thereof is derived from Morus alba.

12. The method of claim 10, wherein the compound or the pharmaceutically acceptable salt thereof exhibits anti-aging or whitening effects.

13. The method of claim 10, wherein the skin disease is a skin aging disease; or a skin disease caused by overproduction of melanin.

14. The method of claim 13, wherein the skin aging disease is one or more selected from the group consisting of skin wrinkles, photosenescence, melanosis, skin pigmentation, skin burns, skin inflammation, and skin cancer.

15. The method of claim 13, wherein the skin disease caused by overproduction of melanin is one or more selected from the group consisting of melasma, freckles, blemishes, age spots, and Ota nevus.

16. The method of claim 10, wherein the cosmetic composition has a formulation selected from the group consisting of skin lotions, skin softeners, skin toners, astringents, milk lotions, moisture lotions, nourishing lotions, massage creams, nourishing creams, moisture creams, hand creams, foundations, essences, nourishing essences, packs, soaps, cleansing foams, cleansing lotions, cleansing creams, body lotions, and body cleansers.

17. A method for preventing or ameliorating a skin disease, comprising administering a foodologically effective amount of a health functional food composition comprising a compound represented by the following Chemical Formula 1 or a pharmaceutically acceptable salt thereof as an active ingredient to a subject in need of preventing or ameliorating the skin disease:

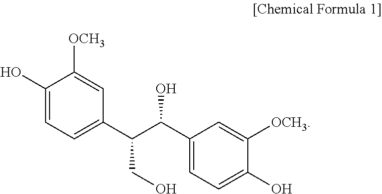 [SMILES: COc1cc([C@@H](O)[C@H](CO)c2ccc(O)c(OC)c2)ccc1O]

18. A method for preventing or ameliorating a skin disease, comprising administering pharmaceutically effective amount of a pharmaceutical composition comprising a compound represented by the following Chemical Formula 1 or a pharmaceutically acceptable salt thereof as an active ingredient to a subject in need of preventing or ameliorating the skin disease: 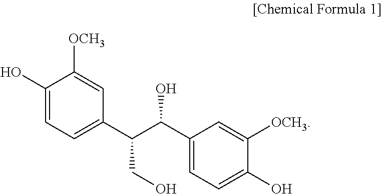 [SMILES: COc1cc([C@@H](O)[C@H](CO)c2ccc(O)c(OC)c2)ccc1O]
